(19) United States
(12) Patent Application Publication
(10) Pub. No.: US 2013/0123741 A1
(43) Pub. Date: May 16, 2013

(54) FLEXIBLE STERILE BAG CONTAINING PHARMACEUTICAL PRODUCTS AND DILUANT SEPARATELY AND METHOD OF MAKING THE SAME

(71) Applicant: GALENOVA INC, St-Hyacinthe (CA)

(72) Inventors: Bertrand BOLDUC, St-Hyacinthe (CA); Jean-Philippe GENTES, Montreal (CA)

(73) Assignee: GALENOVA INC, St-Hyacinthe (CA)

(21) Appl. No.: 13/630,133

(22) Filed: Sep. 28, 2012

Related U.S. Application Data
(60) Provisional application No. 61/559,319, filed on Nov. 14, 2011.

(51) Int. Cl. A61J 1/14; A61P 31/00; A61P 25/04; A61K 31/4468; A61K 31/546

(52) U.S. Cl. 604/416; 514/329; 514/206

(57) ABSTRACT

The present document describes a formulation for the preparation of an injectable composition comprising at least one pH regulator, and pharmaceutical ingredients, to prepare an injectable composition at a predetermined pH value; Also described are containers for combined storage and administration of powdered or liquid formulations and diluents.

### CROSS-REFERENCE TO RELATED APPLICATIONS

This application claims priority of U.S. provisional patent application 61/559,319 filed on Nov. 14, 2011 the specification of which is hereby incorporated by reference.

### BACKGROUND

(a) Field

The subject matter disclosed generally relates to a powder formulation or a liquid formulation, an injectable composition which may, or may not be pH-regulated and/or cosmetic composition and a flexible and sterile container for combined storage and administration of the disclosed powder formulation and diluents.

(b) Related Prior Art

In the medical, the pharmaceutical and the cosmetic fields, it is often necessary to mix two or more separate component such as a mixture of a drug and a diluent. Such mixtures must often be stored separately. In some cases, the mixture is not stable over long periods of time, which can become a problem in the transportation, storage, administration or extamporeneous administration of those mixtures. Such instabilities may also be the case for the mixture of a drug and diluents. Also, some drugs lose their efficacy when stored in liquid diluents and must be stored dry, such as powdered drug stored in a drug vial. Furthermore, some drugs or compounds in solution loose the efficacy when further mixed with diluents. For example, most liquid diluents, such as sterile water, are sterilized by steam sterilization, or autoclaving. The heat generated during such a sterilization procedure may destroy the efficacy of many powdered drugs which is a problem to be avoided. Moreover, the problem is not limited to sterilization procedure but may also stem from the fact that drugs may be unstable in liquid solutions, which cause undesired chemical reactions such as hydrolysis, which may result in the formation of new unwanted complexes, loss of efficacy or drug degradation.

Therefore, there is a need for the mixing of diluant solutions and dry powdered drugs or drugs in solution in a closed and preferably sterile system, to prepare solutions of predetermined concentrations of drugs. Such systems reduce the risks of pathogenic contamination, while also reducing the chance of administering the wrong dose of drug to a patient. Thus, to maintain two or more components separately during storage, there is a need for a quick and easy means for selectively mixing the two or more components, in a closed system, under conditions which may be sterile. It is known to provide a multiple compartment container of flexible plastic sheets with a heat seal dividing the container into two or more compartments. It is also known to provide a container which employs frangible valves between the heat seal to allow for selective communication and mixing between the two or more components stored in the two or more compartments. However, there is a need to provide a bag capable of mixing in a closed-environment a product and a diluent under sterile conditions. Sterile conditions are very important for avoiding contamination.

It is also known to provide a closed system for the separate storage and selective mixing of two components via a device utilizing a preferably plastic junction about the end portions of container and compartment access means to allow for the separate storage and selective mixing of two components such as powdered drug and a liquid diluents.

It is desirable to provide a multiple compartment closed system which does not require additional elements integrated into the container to form the openable valve between the compartments. It is well known to provide a multiple compartment container which has a line of weakness, such as a score line in plastic material, which breaks upon the application of pressure. Tear tabs or tear strips for plastic packagings are also known. Such tear tabs provide ready access to the contents of a container but also involve the use of a relatively complicated seal structure. However, rupturable seal lines may all suffer for the common problems of rupturing before communication between the two compartments is intended. The rupturable seal lines are designed to be weaker than the remainder of the container so that upon the application of force the rupture line breaks first.

It is also desirable to provide a multiple compartment closed container having a selectively openable seal line between the compartment which when closed virtually eliminates the transmission of moisture into one of the compartments from either outside of the container or from the other compartment. Such a requirement may be necessary when, for example, a powdered drug is stored in one of the compartments of the container.

It is also desirable to provide a container having at least a first compartment for receiving a diluent, a second compartment for receiving a powder formulation or another liquid formulation, and an external port extending from the first compartment to the external environment, where the powder formulation or the other liquid formulation has no access from the second compartment to the external environment via the outlet port.

There is therefore a need for a powder formulation, an injectable composition which may or may not be pH-regulated and a low cost flexible and sterile container for combined storage and administration of the disclosed powder formulation and diluents

### SUMMARY

According to an embodiment, there is provided a powder formulation for the preparation of an injectable composition comprising:

at least one pH regulator; and

a pharmaceutical ingredient chosen from an antibiotic, an analgesic, an antipyretic, a local anesthetic, an NSAIDS, an antifungal, an antiviral, an angiotensin-converting enzyme (ACE) inhibitor, an angiotensin II antagonist, an aldosterone antagonist, a β-adrenergic antagonist, an antineoplastic agent, an immunosuppressive drug and a combination thereof;

wherein the pH regulator sets the pH of an injectable composition at a predetermined value from about 3 to about 11.

The pH regulator may be chosen from NaOH, HCl, KOH, acetic acid, acetate buffer an organic acid, a mineral acid, a base, a neutralizing agent, and a combination thereof.

The formulation may be further comprising a buffering agent.

The buffering agent may be chosen from sodium dihydrogenphosphate (Na₂HPO₄), sodium dihydrogenphosphate (NaH₂PO₄), TRIS, sodium bicarbonate, sodium carbonate and a combination thereof.

The pharmaceutical ingredient may be chosen from an antibiotic, an analgesic, an antipyretic, a local anesthetic, an NSAIDS, an antifungal, an antiviral, an angiotensin-converting enzyme (ACE) inhibitor, an angiotensin II antagonist, an aldosterone antagonist, a β-adrenergic antagonist, an antineoplastic agent, a immunosuppressive drug and a combination thereof.

The antibiotic may be chosen from penicillin, a cephalosporin, tetracycline, erythromycin, vancomycin, polymixin, gramicidin, tetracycline, macrolide, chloramphenicol, clindamycin, spectinomycin, sulfonamide and a combination thereof.

The cephalosporin may be chosen from cefazoline, cefoxitine, cefotaxime, cephacetrile, cefadroxyl, cefalexin, cefaloglycin, cefalonium, cefaloradine, cefalothin, cefapirin, Cefatrizine, Cefazaflur, Cefazedone, cefradine, Cefroxadine, Ceftezole, Cefaclor, Cefonicid, Cefprozil, Cefuroxime, Cefuzonam, Cefmetazole, Cefotetan, Cefoxitin, loracarbef; Cephamycins, cefbuperazone, cefmetazole, cefminox, cefotetan, cefoxitin, Cefcapene, Cefdaloxime, Cefdinir, Cefditoren, Cefetamet, Cefixime, Cefmenoxime, Cefodizime, Cefotaxime, Cefovecin, Cefpimizole, Cefpodoxime, Cefteram, Ceftibuten, Ceftiofur, Ceftiolene, Ceftizoxime, Ceftriaxone, Cefoperazone, Ceftazidime, Oxacephems, latamoxef, Cefclidine, Cefepime, Cefluprenam, Cefoselis, Cefozopran, Cefpirome, Cefquinome, flomoxef, Ceftobiprole, and Ceftaroline.

The analgesic may be chosen from morphine, fentanyl, hydromorphone, sufentanil, clonidine, ketamine, sufentanil, oxymorphone, scopolamine, pethidine, piritramide, codeine, dihydrocodeine, ethylmorphine, methadone, loperamide, difenoxin, diphenoxylate, paregoric, powdered opium, laudanum, buprenorphine, Meperidine, Alfentanil, Remifentanil, Sufentanil, Etorphine, oxycodone, oxymorphone, desomorphine, nicomorphine, dipropanoylmorphine, benzylmorphine, tramadol, dextropropoxyphene, Thebaine, Oripavine, Heroin (diacetylmorphine), Nicomorphine, Fentanyl, Alphamethylfentanyl, Sufentanil, Remifentanil, Carfentanyl, Ohmefentanyl, Ketobemidone, MPPP, Allylprodine, Prodine, PEPAP, Propoxyphene, Dextropropoxyphene, Dextromoramide, Bezitramide, Dipipanone, Levomethadyl Acetate (LAAM), Dihydroetorphine, Etorphine, Butorphanol, Nalbuphine, Levorphanol, Levomethorphan, Lefetamine, Meptazinol, Tilidine, and Tapentadol and a combination thereof.

The local anesthetic may be chosen from procaine, amethocaine, cocaine, lidocaine, prilocaine, bupivacaine, levobupivacaine, ropivacaine, mepivacaine, dibucaine, marcaine, sensorcaine, vivacaine, ropivacaine, and chloroprocaine.

The antipyretic may be chosen from an NSAID, aspirin, choline salicylate, magnesium salicylate, and sodium salicylate, acetaminophen, metamizole, nabumetone, nimesulide, phenazone and quinine.

The NSAIDS may be chosen from acetaminophen, acemetacin, aceclofenac, alminoprofen, arnfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine and a combination thereof.

The antifungal may be chosen from natamycin, rimocidin, filipin, nystatin, amphotericin B, candicin, hamycin, miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, abafungin, terbinafine, naftifine, butenafine, anidulafungin, caspofungin, micafungin, polygodial, benzoic acid, ciclopirox, tolnaftate, undecyclenic acid, flucytosine, griseofulvin, haloprogin, sodium bicarbonate (NaHCO3), allicin, tea tree oil, citronella oil, iodine, olive leaf, orange oil, palmarosa oil, patchouli, lemon myrtle, neem seed oil, coconut oil, zinc, selenium and a combination thereof.

The antiviral may be chosen from Oseltamivir (tamiflu), abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, entry inhibitors, famciclovir, fomivirsen, fosamprenavir, foscarnet, fosfonet, fusion inhibitor, ganciclovir, ibacitabine, imunovir, idoxuridine, imiquimod, indinavir, inosine, integrase inhibitor, lamivudine, lopinavir, loviride, maraviroc, moroxydine, methisazone, nelfinavir, nevirapine, nexavir, peginterferon, penciclovir, peramivir, pleconaril, podophyllotoxin, protease inhibitor, raltegravir, ribavirin, rimantadine, ritonavir, pyramidine, saquinavir, stavudine, tea tree oil, tenofovir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir (relenza), zidovudine and a combination thereof.

The angiotensin-converting enzyme (ACE) inhibitor may be chosen from Captopril, Zofenopril, Enalapril, Ramipril, Quinapril, Perindopril, Lisinopril, Benazepril, Imidapril, Zofenopril, Fosinopril, and a combination thereof.

The angiotensin II antagonist may be chosen from Valsartan, Telmisartan, Losartan, Irbesartan, Azilsartan, Olmesartan, and Irbesartan.

The aldosterone antagonist may be chosen from Spironolactone, Eplerenone, Canrenone, Prorenone, Mexrenone, and a combination thereof.

The β-adrenergic antagonist may be chosen from Alprenolol, Bucindolol, Carteolol, Carvedilol, Labetalol, Nadolol, Oxprenolol, Penbutolol, Pindolol, Propranolol, Sotalol, Timolol, Eucommia bark, Acebutolol, Atenolol, Betaxolol, Bisoprolol, Celiprolol, Esmolol, Metoprolol, Nebivolol, Butaxamine, ICI-118,551, SR 59230A, and a combination thereof.

The antineoplastic agent may be chosen from Cyclophosphamide, Mechlorethamine, Uramustine, Melphalan, Chlorambucil, Ifosfamide, Carmustine, Lomustine, Streptozocin, Busulfan, Thiotepa, Cisplatin, Carboplatin, Nedaplatin, Oxaliplatin, Satraplatin, Triplatin tetranitrate, procarbazine, altretamine, dacarbazine, mitozolomide, temozolomide, azathioprine, mercaptopurine, Vincristine, Vinblastine, Vinorelbine, Vindesine, Podophyllotoxin, docetaxel, paclitaxel, irinotecan, topotecan, epipodophyllotoxins, amsacrine, etoposide, etoposide phosphate, teniposide, actinomycin, anthracyclines, doxorubicin, daunorubicin, valrubicin, idarubicin, epirubicin, bleomycin, plicamycin, mitomycin and a combination thereof.

The immunosuppressive drug may be chosen from a glucocorticoid, Methotrexate, Azathioprine, dactinomycin, anthracyclines, mitomycin C, bleomycin, mithramycin, Ciclosporin, Tacrolimus, Sirolimus, an interferon, Mycophenolic acid, Fingolimod, myrriocyn, and a combination thereof.

According to another embodiment, there is provided a container for combined storage and administration of a powder or liquid formulation and diluent comprising:
a first compartment for receiving a diluent comprising an outlet port for providing an access from the first compartment to an external environment, and a first inlet port for providing an access for filling the first compartment; and
a second compartment, adjacent to the first compartment, for containing a powder or liquid formulation, comprising a second inlet port for providing an access for filling the second compartment; and
a breakable seal joining the first and the second compartments.

The container may be further comprising the formulation in the second compartment.

The container may comprise the formulation according to the present invention.

The container may be further comprising the diluent in the first compartment.

The diluent may be chosen from sterilized water, water, saline solution, water with 0.9% NaCl, water with 5% dextrose, sterile liquids, an alcool, polyethylene glycol, oils, sesame oil, soybean oil, saline solutions, aqueous dextrose solutions, aqueous glycerol solutions, aqueous dextrose and glycerol solutions, phosphate buffer, phosphate buffered saline, lactated Ringer's solution and a combination thereof.

The diluent may be sterilized water.

The diluent may be water with 0.9% NaCl.

The diluent may be water with 5% dextrose.

The container may be further comprising a third compartment adjacent to the first compartment or the second compartment, for containing a powder or liquid formulation.

According to another embodiment, there is provided a ready to use container comprising:
a first compartment containing a diluent chosen from sterilized water, water, saline solution, water with 0.9% NaCl, water with 5% dextrose, sterile liquids, an alcool, polyethylene glycol, oils, sesame oil, soybean oil, saline solutions, aqueous dextrose solutions, aqueous glycerol solutions, aqueous dextrose and glycerol solutions, phosphate buffer, phosphate buffered saline, lactated Ringer's solution and a combination thereof, in for receiving said diluent, the first compartment comprising an outlet port for providing an access from the first compartment to an external environment,
a second compartment containing a formulation of the present invention, adjacent to the first compartment, for containing the formulation, and,
a breakable seal joining the first and the second compartments.

The following terms are defined below.

The term “diluent” is intended to mean a diluting agent that is not too viscous to be easily pumped and that is not too dense to easily flow from one particular point to another. The “diluent” may be, without limitations, sterilized water, water, sterile liquids, an alcool, polyethylene glycol, oils (including those of petroleum, animal, vegetable or synthetic), sesame oil, soybean oil, saline solutions, aqueous dextrose solutions, aqueous glycerol solutions, aqueous dextrose and glycerol solutions, phosphate buffer, phosphate buffered salines, lactated Ringer's solution, and the like, and combinations thereof.

The term “pharmaceutical ingredient” is intended to mean a compound that is an active ingredient in the composition of the present invention. The term “pharmaceutical ingredient” is intended to mean any compound, such as for example antibiotics, painkiller, cosmetic ingredients (e.g. collagen), and the like. The term “pharmaceutical ingredient” is broad in its definition and may include glucose, as part of a pharmaceutically administered composition, for instance.

The term “pH-regulated injectable composition” is intended to mean a composition having a powder formulation which when the powder formulation is mixed with the diluents has a predetermined pH value, and ingredients which contain a combination of a weak acid and its salt or a weak base and its salt. The pH is regulated to obtain the preferred pH for the way the drug is meant to be administered.

The term “predetermined value” is intended to mean a value that is established, calculated or determined in advance.

The term “breakable seal” is intended to mean a seal which is sufficiently durable to allow normal handling of the container yet which will peel, to allow separation of the front sheet from the back sheet in the region of the seal, under hydraulic pressure, or the like, applied by manipulating the container, thereby allowing mixing and dispending of the solution container contains.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates the front view of one embodiment of a flexible and sterile container for combined storage and administration of a powder formulation and a diluent;

FIG. 2 illustrates another embodiment of the flexible and sterile container; and

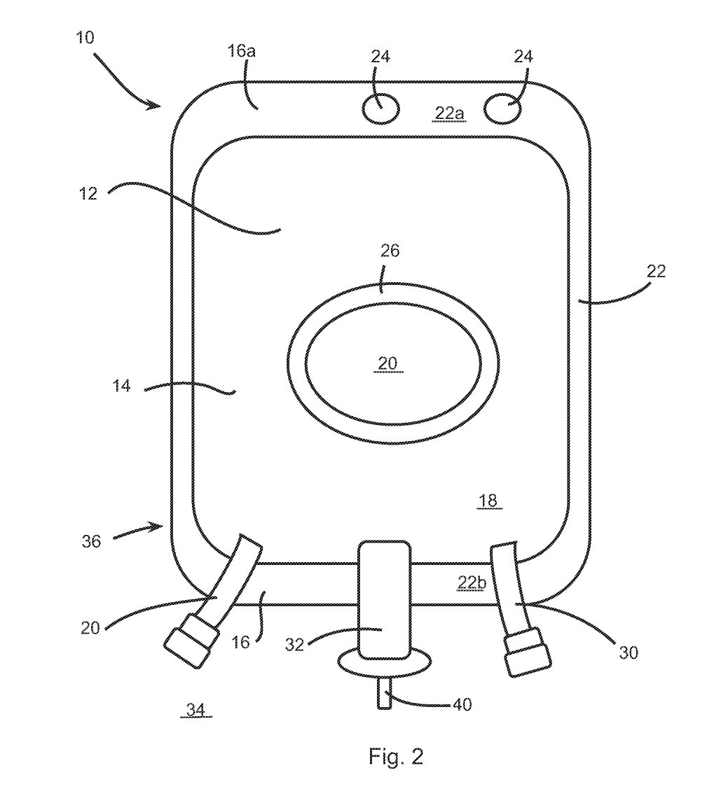

FIG. 3 illustrates another embodiment of the flexible and sterile container.

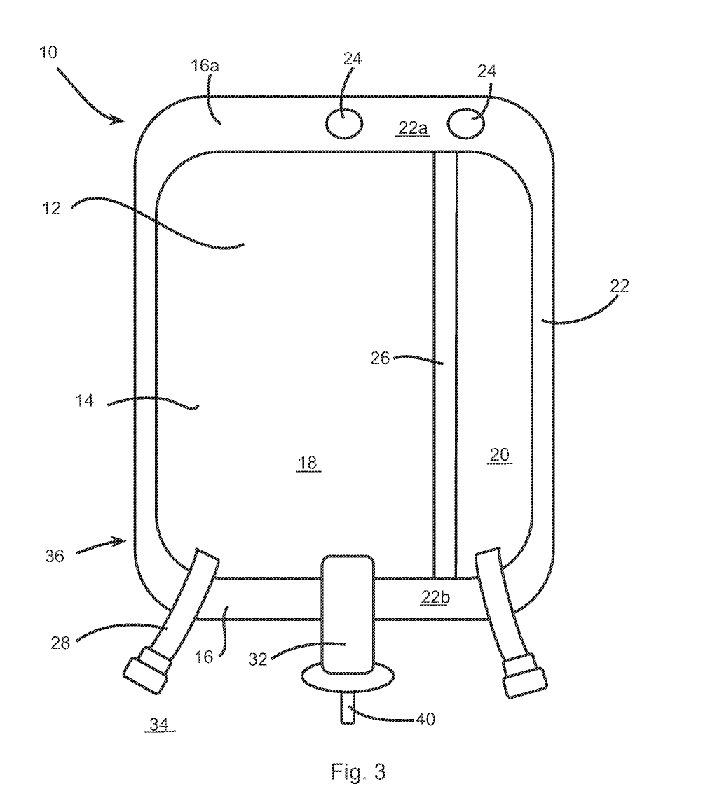

FIG. 4a illustrates another embodiment of the flexible and sterile container.

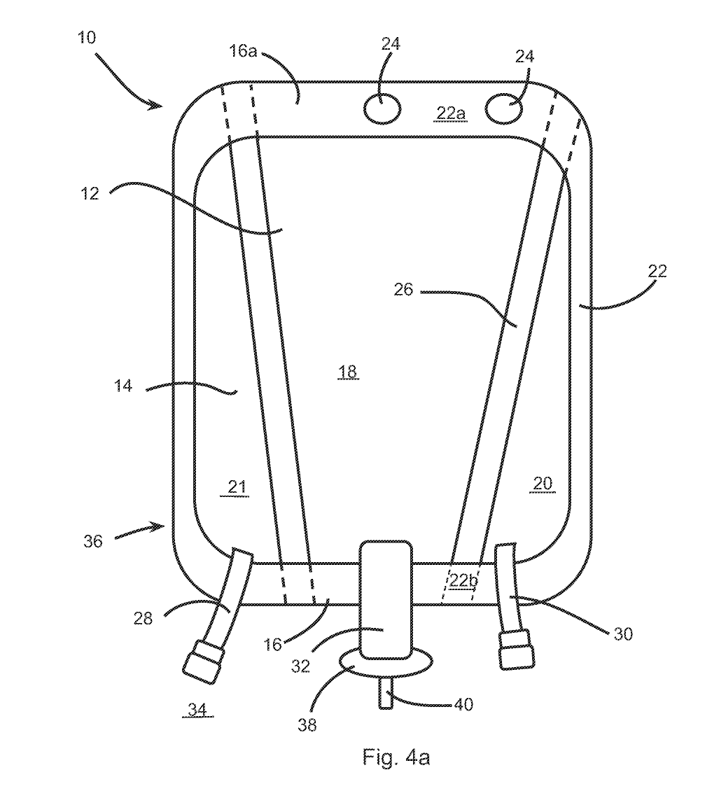

FIG. 4b illustrates another embodiment of the flexible and sterile container.

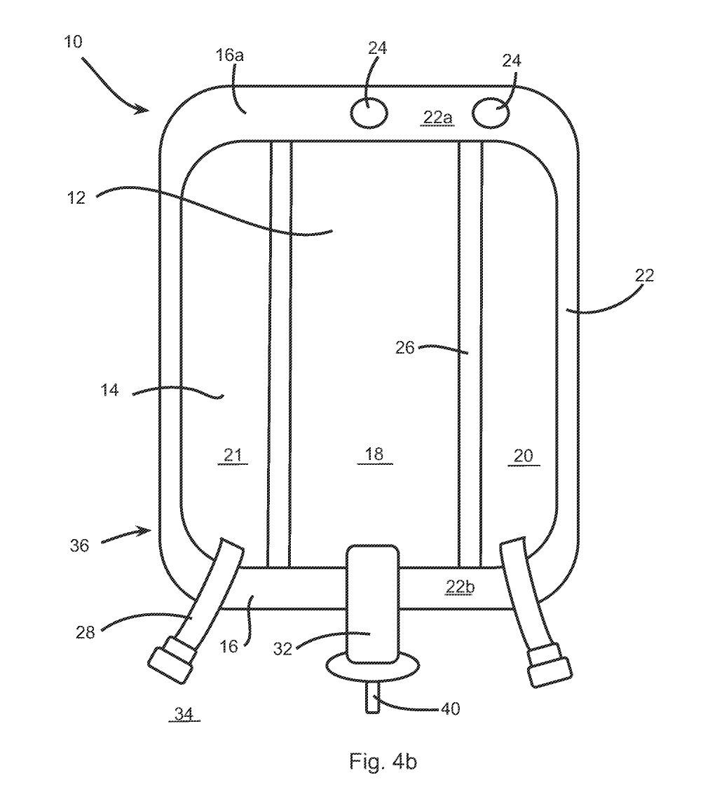

It will be noted that throughout the appended drawings, like features are identified by like reference numerals.

### DETAILED DESCRIPTION OF THE PREFERRED EMBODIMENTS

In embodiments, there is described a powder formulation for the preparation of an injectable composition. The powder formulation according to the present invention may be used to facilitate the formulation of drugs, particularly by improving their stability over longer periods of time. The powder formulation may comprise at least one pH regulator, a pharmaceutical ingredient and excipients. Numerous drugs require the adjustment of the pH of the solution in order to be properly administered, and therefore, when the powder formulation is mixed with a diluent, the pH of the injectable composition has a predetermined value when the at least one pH regulator is present. The predetermined pH value of the injectable composition may be from about 3 to about 11. More specifically, the injectable composition may have a pH from about 7.2 to about 7.5. Most preferably, the pH of the injectable composition is about 7.4, which approaches the physiological pH of 7.365. Therefore, the pH of the injectable composition may correspond to the physiological pH, while preserving the ingredients in a stable solution for a predetermined period of time before the injection of the composition. However, according to another embodiment, the pH of the injectable composition may be any suitable pH for the proper administration of certain drugs. Not all drugs need or can be administered at a physiological pH, and the scope of the present invention also includes those pH values and range suitable for administration of such drugs. Therefore, the range of suitable pH may be from about 3 to about 11.

In the powder formulation as described above, the pH regulator may include, without limitations, NaOH, HCl, KOH, organic acids, mineral acids, bases, neutralizing agents, sodium dihydrogenphosphate (Na₂HPO₄), sodium dihydrogenphosphate (NaH₂PO₄), TRIS, sodium bicarbonate, sodium carbonate acetic acid, acetate buffer or any suitable buffer characterized by a weak acid or a weak base and its salt, and the like. The raw powders (pH regulators and pharmaceutical ingredients) are generally mixed dry in a mixer.

According to another embodiment, the powder formulation may comprise a buffering agent. Examples of buffering agents include, without limitations sodium dihydrogenphosphate (Na₂HPO₄), sodium dihydrogenphosphate (NaH₂PO₄), TRIS, sodium bicarbonate, sodium carbonate, acetate buffer and a combination thereof. However, the buffering agent may also be present in the diluent with which the powder formulation of the present invention will be mixed.

According to another embodiment, the particle size is important to the performance and appearance of the powder formulation. For example, the particle size may be important in the dissolution speed of the powder. Furthermore, the injectable composition of the present invention may be iso-osmotic; and therefore, the powder or liquid formulation according to the present invention will be formulated accordingly as is known in the art in order to be iso-osmotic.

Moreover, in the powder formulation, the pharmaceutical ingredient may include, without limitations;

an antibiotic, such as for example, without limitations, penicillin, cephalosporins which include cefazoline, cefoxitine, cefotaxime, cephacetrile, cefadroxyl, cefalexin, cefaloglycin, cefalonium, cefaloradine, cefalothin, cefapirin, Cefatrizine, Cefazaflur, Cefazedone, cefradine, Cefroxadine, Ceftezole, Cefaclor, Cefonicid, Cefprozil, Cefuroxime, Cefuzonam, Cefmetazole, Cefotetan, Cefoxitin, loracarbef; Cephamycins, cefbuperazone, cefmetazole, cefminox, cefotetan, cefoxitin, Cefcapene, Cefdaloxime, Cefdinir, Cefditoren, Cefetamet, Cefixime, Cefmenoxime, Cefodizime, Cefotaxime, Cefovecin, Cefpimizole, Cefpodoxime, Cefteram, Ceftibuten, Ceftiofur, Ceftiolene, Ceftizoxime, Ceftriaxone, Cefoperazone, Ceftazidime, Oxacephems, latamoxef, Cefclidine, Cefepime, Cefluprenam, Cefoselis, Cefozopran, Cefpirome, Cefquinome, flomoxef, Ceftobiprole, and Ceftaroline. Other antibiotics include tetracycline, erythromycin, vancomycin, polymixin, gramicidin, tetracycline, macrolide, chloramphenicol, clindamycin, spectinomycin, sulfonamide, and the like;

an analgesic such as for example, without limitations, morphine, fentanyl, hydromorphone, sufentanil, clonidine, ketamine, sufentanil, oxymorphone, scopolamine, pethidine, piritramide, codeine, dihydrocodeine, ethylmorphine, hydrocodone, methadone, loperamide, difenoxin, diphenoxylate, paregoric, powdered opium, laudanum, buprenorphine, Meperidine, Alfentanil, Remifentanil, Sufentanil, Etorphine, oxycodone, oxymorphone, desomorphine, nicomorphine, dipropanoylmorphine, benzylmorphine, tramadol, dextropropoxyphene, Thebaine, Oripavine, Heroin (diacetylmorphine), Nicomorphine, Fentanyl, Alphamethylfentanyl, Sufentanil, Remifentanil, Carfentanyl, Ohmefentanyl, Ketobemidone, MPPP, Allylprodine, Prodine, PEPAP, Propoxyphene, Dextropropoxyphene, Dextromoramide, Bezitramide, Dipipanone, Levomethadyl Acetate (LAAM), Dihydroetorphine, Etorphine, Butorphanol, Nalbuphine, Levorphanol, Levomethorphan, Lefetamine, Meptazinol, Tilidine, and Tapentadol and the like;

an antipyretic, which include NSAID as defined below, as well as aspirin, and related salicylates like choline salicylate, magnesium salicylate, and sodium salicylate, paracetamol (Acetaminophen), metamizole, nabumetone, nimesulide, phenazone and quinine;

A local anesthetic, such as for example procaine, amethocaine, cocaine, lidocaine (also known as Lignocaine), prilocaine, bupivacaine, levobupivacaine, ropivacaine, mepivacaine, dibucaine, marcaine, sensorcaine, vivacaine, ropivacaine, chloroprocaine; an NSAIDS, such as for example, without limitations, acetaminophen, acemetacin, aceclofenac, alminoprofen, arnfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine, prodrugs thereof, and the like;

an antifungal, such as for example, without limitations, natamycin, rimocidin, filipin, nystatin, amphotericin B, candicin, hamycin, miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, abafungin, terbinafine, naftifine, butenafine, anidulafungin, caspofungin, micafungin, polygodial, benzoic acid, ciclopirox, tolnaftate, undecyclenic acid, flucytosine, griseofulvin, haloprogin, sodium bicarbonate (NaHCO₃), allicin, tea tree oil, citronella oil, iodine, olive leaf, orange oil, palmarosa oil, patchouli, lemon myrtle, neem seed oil, coconut oil, zinc, selenium and the like; and

an antiviral, such as for example, without limitations, Oseltamivir (tamiflu), abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, entry inhibitors, famciclovir, fomivirsen, fosamprenavir, foscarnet, fosfonet, fusion inhibitor, ganciclovir, ibacitabine, imunovir, idoxuridine, imiquimod, indinavir, inosine, integrase inhibitor, lamivudine, lopinavir, loviride, maraviroc, moroxydine, methisazone, nelfinavir, nevirapine, nexavir, peginterferon, penciclovir, peramivir, pleconaril, podophyllotoxin, protease inhibitor, raltegravir, ribavirin, rimantadine, ritonavir, pyramidine, saquinavir, stavudine, tea tree oil, tenofovir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir (relenza), zidovudine and the like.

an angiotensin-converting enzyme (ACE) inhibitor, such as for example Captopril, Zofenopril, Enalapril, Ramipril, Quinapril, Perindopril, Lisinopril, Benazepril, Imidapril, Zofenopril, and Fosinopril.

an angiotensin II antagonist, such as for example Valsartan, Telmisartan, Losartan, Irbesartan, Azilsartan, Olmesartan, Irbesartan.

an aldosterone antagonist, such as for example Spironolactone, Eplerenone, Canrenone, Prorenone, and Mexrenone.

a β-adrenergic antagonist, such as for example Alprenolol, Bucindolol, Carteolol, Carvedilol, Labetalol, Nadolol, Oxprenolol, Penbutolol, Pindolol, Propranolol, Sotalol, Timolol, Eucommia bark, Acebutolol, Atenolol, Betaxolol, Bisoprolol, Celiprolol, Esmolol, Metoprolol, Nebivolol, Butaxamine, ICI-118,551, and SR 59230A.

an antineoplastic agent, such as for example Cyclophosphamide, Mechlorethamine, Uramustine, Melphalan, Chlorambucil, Ifosfamide, Carmustine, Lomustine, Streptozocin, Busulfan, Thiotepa, Cisplatin, Carboplatin, Nedaplatin, Oxaliplatin, Satraplatin, Triplatin tetranitrate, procarbazine, altretamine, dacarbazine, mitozolomide, temozolomide, azathioprine, mercaptopurine, Vincristine, Vinblastine, Vinorelbine, Vindesine, Podophyllotoxin, docetaxel, paclitaxel, irinotecan, topotecan, epipodophyllotoxins, amsacrine, etoposide, etoposide phosphate, teniposide, actinomycin, anthracyclines, doxorubicin, daunorubicin, valrubicin, idarubicin, epirubicin, bleomycin, plicamycin, and mitomycin.

an immunosuppressive drug, such as for example a glucocorticoid, Methotrexate, Azathioprine, dactinomycin, anthracyclines, mitomycin C, bleomycin, mithramycin, Ciclosporin, Tacrolimus, Sirolimus, an interferon, Mycophenolic acid, Fingolimod, and myrriocyn.

Also, the diluent mixed with the powder formulation to obtain an injectable composition of a predetermined pH value may be, without limitations, sterilized water, water, sterile liquids, an alcohol, polyethylene glycol, oils (including those of petroleum, animal, vegetable or synthetic), sesame oil, soybean oil, saline solutions, aqueous dextrose solutions, aqueous glycerol solutions, aqueous dextrose and glycerol solutions, phosphate buffer, phosphate buffered saline, lactate Ringer, or combinations thereof, and the like.

The diluent may comprise a solute composition which upon dissolution of the pharmaceutical ingredient and pH regulator will for a suitable solution for intravenous injection, or any other type of injection, as known in the art. Moreover, the pH regulator may be found in the form of a powder with the pharmaceutical ingredient, or it may be included in the diluent.

The diluent may be sterilized water. Preferably, the diluents may also be 0.9% NaCl or 5% dextrose. The diluents may be inert, compatible with the powder formulation or relatively cheap.

Secondly, there is also described an injectable composition comprising the powder formulation as described above and a diluent. It is to be noted that the powder formulation mixed with the diluent has a predetermined pH value. The predetermined pH value of the injectable composition may be in the pH range from about 7 to 8. More specifically, the injectable composition is from about 7.2 to about 7.5. Most preferably, the pH of the injectable composition is about 7.4, which approaches the physiological pH of 7.365. Therefore, the pH of the injectable composition may correspond to the physiological pH, while preserving the ingredients in a stable solution for a predetermined period of time before the injection of the composition. However, according to another embodiment, the pH of the injectable composition may be any suitable pH for the proper administration of certain drugs. Not all drugs need or can be administered at a physiological pH, and the scope of the present invention also includes those pH values and range suitable for administration of such drugs. Therefore, the range of suitable pH may be from about 3 to about 11.

Additionally, the diluent mixed with the powder formulation to obtain an injectable composition of a predetermined pH value may be, without limitations, sterilized water, water, sterile liquids, an alcool, polyethylene glycol, oils (including those of petroleum, animal, vegetable or synthetic), sesame oil, soybean oil, saline solutions, aqueous dextrose solutions, aqueous glycerol solutions, aqueous dextrose and glycerol solutions, phosphate buffer, phosphate buffered salines, and lactated Ringer's solution, and the like. The diluent may be sterilized water. Preferably, the diluent may be saline (e.g. 0.9% NaCl) or dextrose 5%.

There is also described a cosmetic composition comprising the powder formulation as described above and a diluent. It is to be noted that the powder formulation mixed with the diluent has a predetermined pH value. The predetermined pH value of the cosmetic composition may be in the pH range from about 7 to about 8. More specifically, the cosmetic composition may have a pH from about 7.2 to about 7.5. Most preferably, the pH of the cosmetic composition is about 7.4, which approaches the physiological pH of 7.365. Therefore, the pH of the cosmetic composition may correspond to the physiological pH, while preserving the ingredients in a stable solution for a predetermined period of time before the injection of the composition. However, according to another embodiment, the pH of the injectable composition may be any suitable pH for the proper administration of certain drugs. Not all drugs need or can be administered at a physiological pH, and the scope of the present invention also includes those pH values and range suitable for administration of such drugs. Therefore, the range of suitable pH may be from about 3 to about 11.

Also, the diluent mixed with the powder formulation to obtain the cosmetic composition of a predetermined pH value may be, without limitations, sterilized water, water, sterile liquids, an alcohol, polyethylene glycol, oils (including those of petroleum, animal, vegetable or synthetic), sesame oil, soybean oil, saline solutions, aqueous dextrose solutions, aqueous glycerol solutions, aqueous dextrose and glycerol solutions, phosphate buffer, phosphate buffered salines, lactated Ringer's solution, or combinations thereof, and the like. The diluent may be sterilized water. Preferably, the diluent may be saline (e.g. 0.9% NaCl) or dextrose 5%.

According to another embodiment of the present invention, the formulations described above could also be liquid solutions, and could comprise all the described ingredients. Drugs may be stable in liquid formulations, but may become unstable only when mixed with certain elements. Their stability may therefore be preserved until treatment or administration is required, by separating them from the diluents.

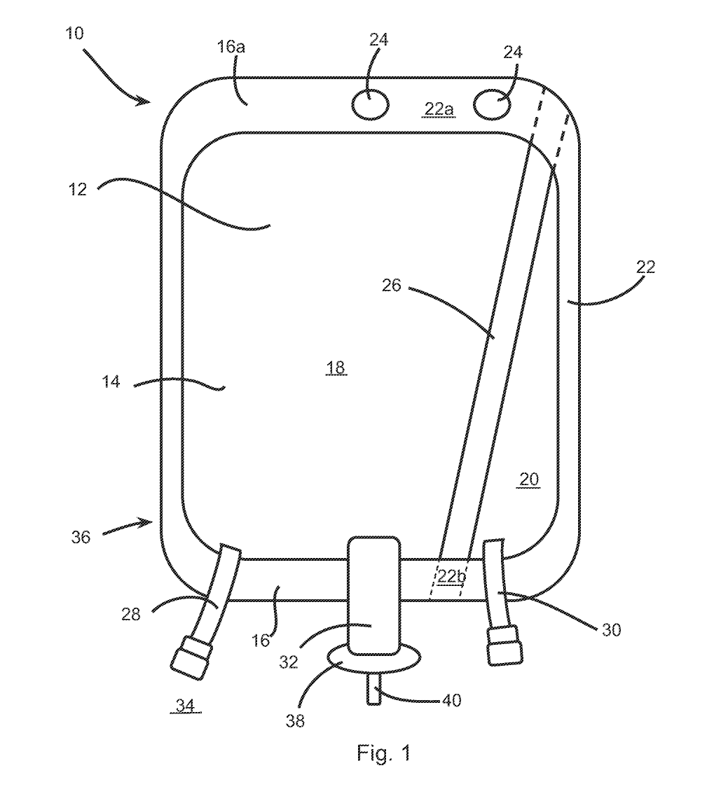

Referring now to the drawings, and more particularly to FIG. 1, there is shown a flexible and sterile container 10. The container 10 may be formed of a front sheet 12 and a back or rear sheet 14 (not shown). The front and back sheets may be constructed of a single layer of flexible material or multi-layer laminates of flexible material. The front and rear sheets 12 and 14 forming the container 10 can be provided separately and then sealed together at their common peripheral edge 22, forming an edge seal 16 which extends around the entire periphery of the container. Such peripheral seals may vary in configuration and width. A patterned seal, such that depicted on the top seal portion 16a in FIG. 1 may be used to provide grasping areas for the user to handle the container 10 and for the attachment of the container to, for example, a common support stand via openings 24.

Alternatively, the front sheet 12 and the rear sheet 14 can be formed from a single sheet which is subsequently folded-over and sealed by means of a heat seal which extends around the peripheral portion of the container 10. The sealed-together sheets 12 and 14 (front sheet 12 and rear sheet 14) are referred to herein as the body 36 of the container 10.

The container 10 is partitioned into two separate compartments; a first compartment 18 and a second compartment 20, each of which is preferably sterile. The first and second compartment 18 and 20 are separated by one another by a breakable seal 26. The breakable seal 26 extends between the peripheral edge 22 of the container 10, i.e, in FIG. 1, between the upper edge 22a and the lower edge 22b, joining the front sheet 12 and the rear sheet 14. According to another embodiment, the breakable seal 26 may extend between any of the edges of peripheral edge 22. More preferably, the breakable seal 26 diagonally extends between any of the edges of peripheral edge 22 for the pressure to be appropriately distributed within the container 10 and prevent rupturing.

As described above, the breakable seal 26 is sufficiently durable to allow normal handling of the container yet which will peel, allowing separation of the front sheet from the back sheet in the region of the seal, under hydraulic pressure, or any other form of pressure, applied by manipulating the container 10, thereby allowing mixing and dissolution of the container contents.

The breakable seal 26 may be obtained by a heat sealing process which is performed with varying length of time, temperatures, and pressures. According to some embodiments, the peripheral edge seal 16 is significantly stronger than the breakable seal 26 and will not be ruptured by pressures generated to separate the breakable seal 26.

The container 10 also provides the first compartment 18 with a principal outlet port 32 which is sealed in the edge seal 16 for providing an access from the interior of the first compartment 18 to the external environment 34. The outlet port 32 extends from the first compartment 18 toward the external environment. The specific configuration of the outlet port 32 for providing an access from the interior of the first compartment 18 to the external environment 14 prevents the powder formulation inserted in the second compartment 18 to enter the outlet port 32, which would distort the concentrations and prevent the solution being used.

In the container 10, the first compartment 18 is preferably filled with a diluent and the second compartment 20 is preferably filled with the powder formulation or the liquid formulation as described above. However, the diluents or powder/liquid formulations may be inverted in the compartments 18 or 20, as may be required. The compartments 18 or 20 may be of same or different sizes, as may also be required as embodiments of the present invention. The diluent may be, without limitations, sterilized water, water, sterile liquids, an alcohol, polyethylene glycol, oils (including those of petroleum, animal, vegetable or synthetic), sesame oil, soybean oil, saline solutions, aqueous dextrose solutions, aqueous glycerol solutions, aqueous dextrose and glycerol solutions, phosphate buffer, phosphate buffered salines, lactated Ringer's solution, or combinations thereof, and the like, and combinations thereof. The first compartment 18 of the container 10 may have a first inlet port 28 which is sealed in the edge seal 16 for providing an access from the external environment 34 to the interior of the first compartment 18. The first inlet port 28 extends from the first compartment 18 toward the external environment. In addition, the second compartment 20 has a second inlet port 30 which is sealed in the edge seal 16 for providing an access from the external environment 34 to the interior of the second compartment 20. Also, the second outlet port 30 extends downwardly from the second compartment 20 toward the external environment. The inlet ports 28 and 30 of the first and the second compartments 18 and 20 may allow the insertion of the powder formulation in the second compartment 20 and the insertion of the diluents in the first compartment 18. The inlet ports 28 and 30 may be cut in the manufacture of the container 10.

According to one embodiment, the outlet port 32 of the container 10 may comprise a body portion and a nozzle 40 which is configured for attachment to a standard fluid administration device. A cap (not shown), may be provided to cover the nozzle 40 and maintain sterility. The cap may be removed just prior to attachment of a standard fluid administration device to the outlet port 32.

According to another embodiment, the container 10 may also comprise an third compartment 21, which may be adjacent to the first or the second compartments 18 or 20. The third compartment 21 may be used when 2 powder formulations, 2 liquid formulations, or 1 powder and 1 liquid formulation are incompatible and would prevent the long term storage of the formulations of the present invention.

The material employed in the front and rear sheets 12 and 14 of the container 10 may be selected based on the material to be stored in the first and second compartment 18 and 20 of the container 10. Preferably, at least one sheet of the front and rear sheets 12 and 14 is transparent to allow the contents of the container 10 to be visually inspected to ascertain complete dissolution of the powder and to allow the level of the solution in the container 10 to be seen during dispensing. Suitable materials for fabrication of the transparent sheet are typically single-layer and multi-layer laminated polymer films.

In particular, whether constructed of a single layer or a multi-layer laminated polymer film, the materials comprising the front 12 and the rear 14 sheets of the container 10 are chosen for their clarity and transparency. The composition of the front 12 and rear 14 sheets of the container 10 may be polyethylenes, polypropylenes, a composition of polyethylenes mixed with polypropylenes, ethylene-vinyl acetate, polyvynil chloride, plastics, silicone membreanes, plastic derivatives, and any other suitable flexible material that may withhold the product.

The present invention will be more readily understood by referring to the following examples which are given to illustrate the invention rather than to limit its scope.

### EXAMPLE 1

### Epidural Pain Management

Epidural analgesia, a form of regional analgesia involves injection of drugs through a catheter placed into the epidural space. The injection can cause both a loss of sensation and a loss of pain. A patient receiving an epidural for pain relief typically receives a combination of local anasthetics and opioids. Common local anasthetics includes lidocaine, bupivacaine, ropivacaine and chloroprocaine. Common opioids include morphine, fentanyl, sufentanil, ketamine and sufentanil.

For example, the container 10 for storing powder formulation and diluents may be used for performing such an epidural analgesia. Indeed, in this specific case, the first compartment 18 of the container 10 is preferably filled with a diluent, such as sterilized water, and the second compartment 20 is preferably filled with the powder formulation. The powder formulation may include at least NaOH as the pH-regulator and may also include at least bupivacaine (from about 0,25 mg/ml to about 1,5 mg/ml) and fentanyl (from about 0,25 pg/ml to about 10 μg/ml) as the pharmaceutical ingredient. When an external pressure pushes on the body 36 of the container 10 and breaks the breakable seal 26 separating the first and the second compartments 18 and 20, the powder formulation is mixed with the diluent, the pH of the formed injectable composition has a predetermined value. The predetermined pH value of the injectable composition is in a pH range from about 4 to about 6.5.

Then the inlet port 32 of the first compartment allows evacuation of the injectable composition toward the external environment, or a standard fluid administration device.

### EXAMPLE 2

### Antibacterial Injectable Solution

A Cefazoline containing bag is prepared by including 1 gram of cefazoline (as cefazoline sodium) and 48 mg of sodium (as sodium chloride, NaCl) into the second compartment 20. The first compartment 10 is filled with 99 ml of a sodium chloride 0,9% solution for injection. Upon reconstitution by breaking the seal between the two compartments, the reconstituted injectable solution comprises 1% Cefazoline in saline solution.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A powder formulation for the preparation of an injectable composition comprising:
at least one pH regulator; and
a pharmaceutical ingredient chosen from an antibiotic, an analgesic, an antipyretic, a local anesthetic, an NSAIDS, an antifungal, an antiviral, an angiotensin-converting enzyme (ACE) inhibitor, an angiotensin II antagonist, an aldosterone antagonist, a β-adrenergic antagonist, an antineoplastic agent, an immunosuppressive drug and a combination thereof;
wherein the pH regulator sets the pH of an injectable composition is at a predetermined value from about 3 to about 11.

2. The formulation of claim 1, wherein the pH regulator is chosen from NaOH, HCl, KOH, acetic acid, acetate buffer an organic acid, a mineral acid, a base, a neutralizing agent, and a combination thereof.

3. The formulation of claim 1, further comprising a buffering agent chosen from sodium dihydrogenphosphate (Na₂HPO₄), sodium dihydrogenphosphate (NaH₂PO₄), TRIS, sodium bicarbonate, sodium carbonate and a combination thereof.

4. The formulation of claim 1, wherein the antibiotic is chosen from penicillin, cephalosporin, tetracycline, erythromycin, vancomycin, polymixin, gramicidin, tetracycline, macrolide, chloramphenicol, clindamycin, spectinomycin, sulfonamide and a combination thereof.

5. The formulation of claim 4, wherein said cephalosporin is chosen from cefazoline, cefoxitine, cefotaxime, cephacetrile, cefadroxyl, cefalexin, cefaloglycin, cefalonium, cefaloradine, cefalothin, cefapirin, Cefatrizine, Cefazaflur, Cefazedone, cefradine, Cefroxadine, Ceftezole, Cefaclor, Cefonicid, Cefprozil, Cefuroxime, Cefuzonam, Cefmetazole, Cefotetan, Cefoxitin, loracarbef; Cephamycins, cefbuperazone, cefmetazole, cefminox, cefotetan, cefoxitin, Cefcapene, Cefdaloxime, Cefdinir, Cefditoren, Cefetamet, Cefixime, Cefmenoxime, Cefodizime, Cefotaxime, Cefovecin, Cefpimizole, Cefpodoxime, Cefteram, Ceftibuten, Ceftiofur, Ceftiolene, Ceftizoxime, Ceftriaxone, Cefoperazone, Ceftazidime, Oxacephems, latamoxef, Cefclidine, Cefepime, Cefluprenam, Cefoselis, Cefozopran, Cefpirome, Cefquinome, flomoxef, Ceftobiprole, and Ceftaroline.

6. The formulation of claim 1, wherein the analgesic is chosen from morphine, fentanyl, hydromorphone, sufentanil, clonidine, ketamine, sufentanil, oxymorphone, scopolamine, pethidine, piritramide, codeine, dihydrocodeine, ethylmorphine, hydrocodone, methadone, loperamide, difenoxin, diphenoxylate, paregoric, powdered opium, laudanum, buprenorphine, Meperidine, Alfentanil, Remifentanil, Sufentanil, Etorphine, oxycodone, oxymorphone, desomorphine, nicomorphine, dipropanoylmorphine, benzylmorphine, tramadol, dextropropoxyphene, Thebaine, Oripavine, Heroin (diacetylmorphine), Nicomorphine, Fentanyl, Alphamethylfentanyl, Sufentanil, Remifentanil, Carfentanyl, Ohmefentanyl, Ketobemidone, MPPP, Allylprodine, Prodine, PEPAP, Propoxyphene, Dextropropoxyphene, Dextromoramide, Bezitramide, Dipipanone, Levomethadyl Acetate (LAAM), Dihydroetorphine, Etorphine, Butorphanol, Nalbuphine, Levorphanol, Levomethorphan, Lefetamine, Meptazinol, Tilidine, and Tapentadol and a combination thereof.

7. The formulation of claim 1, wherein the local anesthetic is chosen from procaine, amethocaine, cocaine, lidocaine, prilocaine, bupivacaine, levobupivacaine, ropivacaine, mepivacaine, dibucaine, marcaine, sensorcaine, vivacaine, ropivacaine, and chloroprocaine.

8. The formulation of claim 1, wherein the antipyretic is chosen from an NSAID, aspirin, choline salicylate, magnesium salicylate, and sodium salicylate, acetaminophen, metamizole, nabumetone, nimesulide, phenazone and quinine.

9. The formulation of claim 1, wherein the NSAIDS is chosen from acetaminophen, acemetacin, aceclofenac, alminoprofen, arnfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine and a combination thereof.

10. The formulation of claim 1, wherein the antifungal is chosen from natamycin, rimocidin, filipin, nystatin, amphotericin B, candicin, hamycin, miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, abafungin, terbinafine, naftifine, butenafine, anidulafungin, caspofungin, micafungin, polygodial, benzoic acid, ciclopirox, tolnaftate, undecyclenic acid, flucytosine, griseofulvin, haloprogin, sodium bicarbonate (NaHCO3), allicin, tea tree oil, citronella oil, iodine, olive leaf, orange oil, palmarosa oil, patchouli, lemon myrtle, neem seed oil, coconut oil, zinc, selenium and a combination thereof.

11. The formulation of claim 1, wherein the antiviral is chosen from Oseltamivir (tamiflu), abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, entry inhibitors, famciclovir, fomivirsen, fosamprenavir, foscarnet, fosfonet, fusion inhibitor, ganciclovir, ibacitabine, imunovir, idoxuridine, imiquimod, indinavir, inosine, integrase inhibitor, lamivudine, lopinavir, loviride, maraviroc, moroxydine, methisazone, nelfinavir, nevirapine, nexavir, peginterferon, penciclovir, peramivir, pleconaril, podophyllotoxin, protease inhibitor, raltegravir, ribavirin, rimantadine, ritonavir, pyramidine, saquinavir, stavudine, tea tree oil, tenofovir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir (relenza), zidovudine and a combination thereof.

12. The formulation of claim 1, wherein the angiotensin-converting enzyme (ACE) inhibitor is chosen from Captopril, Zofenopril, Enalapril, Ramipril, Quinapril, Perindopril, Lisinopril, Benazepril, Imidapril, Zofenopril, Fosinopril, and a combination thereof.

13. The formulation of claim 1, wherein the angiotensin II antagonist is chosen from Valsartan, Telmisartan, Losartan, Irbesartan, Azilsartan, Olmesartan, and Irbesartan.

14. The formulation of claim 1, wherein the aldosterone antagonist is chosen from Spironolactone, Eplerenone, Canrenone, Prorenone, Mexrenone, and a combination thereof.

15. The formulation of claim 1, wherein the β-adrenergic antagonist is chosen from Alprenolol, Bucindolol, Carteolol, Carvedilol, Labetalol, Nadolol, Oxprenolol, Penbutolol, Pindolol, Propranolol, Sotalol, Timolol, Eucommia bark, Acebutolol, Atenolol, Betaxolol, Bisoprolol, Celiprolol, Esmolol, Metoprolol, Nebivolol, Butaxamine, ICI-118,551, SR 59230A, and a combination thereof.

16. The formulation of claim 1, wherein the antineoplastic agent is chosen from Cyclophosphamide, Mechlorethamine, Uramustine, Melphalan, Chlorambucil, Ifosfamide, Carmustine, Lomustine, Streptozocin, Busulfan, Thiotepa, Cisplatin, Carboplatin, Nedaplatin, Oxaliplatin, Satraplatin, Triplatin tetranitrate, procarbazine, altretamine, dacarbazine, mitozolomide, temozolomide, azathioprine, mercaptopurine, Vincristine, Vinblastine, Vinorelbine, Vindesine, Podophyllotoxin, docetaxel, paclitaxel, irinotecan, topotecan, epipodophyllotoxins, amsacrine, etoposide, etoposide phosphate, teniposide, actinomycin, anthracyclines, doxorubicin, daunorubicin, valrubicin, idarubicin, epirubicin, bleomycin, plicamycin, mitomycin and a combination thereof.

17. The formulation of claim 1, wherein the immunosuppressive drug is chosen from a glucocorticoid, Methotrexate, Azathioprine, dactinomycin, anthracyclines, mitomycin C, bleomycin, mithramycin, Ciclosporin, Tacrolimus, Sirolimus, an interferon, Mycophenolic acid, Fingolimod, myrriocyn, and a combination thereof.

18. A container for combined storage and administration of a powder or liquid formulation and diluent comprising:
a first compartment for receiving a diluent comprising an outlet port for providing an access from the first compartment to an external environment, and a first inlet port for providing an access for filling the first compartment; and
a second compartment, adjacent to the first compartment, for containing a powder or liquid formulation, comprising a second inlet port for providing an access for filling the second compartment; and
a breakable seal joining the first and the second compartments.

19. The container of claim 18, further comprising a third compartment adjacent to the first compartment or the second compartment, for containing a powder or liquid formulation.

20. A ready to use container comprising:
a first compartment containing a diluent chosen from sterilized water, water, saline solution, water with 0.9% NaCl, water with 5% dextrose, sterile liquids, an alcool, polyethylene glycol, oils, sesame oil, soybean oil, saline solutions, aqueous dextrose solutions, aqueous glycerol solutions, aqueous dextrose and glycerol solutions, phosphate buffer, phosphate buffered saline, lactated Ringer's solution and a combination thereof, in for receiving said diluent, said first compartment comprising an outlet port for providing an access from the first compartment to an external environment,
a second compartment containing a formulation of claim 1, adjacent to the first compartment, for containing said formulation, and,
a breakable seal joining the first and the second compartments.
